(19) United States
(12) Patent Application Publication
(10) Pub. No.: US 2025/0025143 A1
(43) Pub. Date: Jan. 23, 2025

(54) FASTENING DEVICE AND RELATED METHODS OF USE

(71) Applicant: Boston Scientific Scimed, Inc., Maple Grove, MN (US)

(72) Inventors: Paul SMITH, Smithfield, RI (US); Jeffrey Bean, Fitchburg, MA (US); John GOLDEN, Norton, MA (US); Kenneth Keene, Winchester, MA (US); Douglas Melanson, Natick, MA (US)

(73) Assignee: Boston Scientific Scimed, Inc., Maple Grove, MN (US)

(21) Appl. No.: 18/907,806

(22) Filed: Oct. 7, 2024

Related U.S. Application Data
(63) Continuation of application No. 16/936,862, filed on Jul. 23, 2020, now Pat. No. 12,137,888.
(60) Provisional application No. 62/877,958, filed on Jul. 24, 2019.

(51) Int. Cl. A61B 17/00; A61B 1/00; A61B 17/072

(52) U.S. Cl. CPC A61B 17/00234; A61B 1/00133; A61B 1/0014; A61B 17/072; A61B 2017/00296; A61B 2017/00398; A61B 2017/00473; A61B 2017/00862; A61B 2017/07257

(57) ABSTRACT

A medical assembly may comprise an end effector; a sheath; and a device coupler. The device coupler may include a proximal support, a distal support, and a coupler arm extending between the proximal support and the distal support. The coupler arm may include a surface configured to accommodate a shape of a medical device resting upon the coupler arm. The end effector may be attached to the distal support, and the proximal support may be attached to the sheath.

### CROSS-REFERENCE TO RELATED APPLICATIONS

This application is a continuation of U.S. Nonprovisional application Ser. No. 16/936,862, filed on Jul. 23, 2020, which claims the benefit of priority from U.S. Provisional Application No. 62/877,958, filed Jul. 24, 2019, each of which is incorporated by reference herein in its entirety.

### TECHNICAL FIELD

The present disclosure relates generally to tissue stapling. More particularly, at least some embodiments of the present disclosure relate to a fastening mechanism for a medical device, for example an endoscopic stapler, and related methods of using the fastening mechanism.

### BACKGROUND

Stapling is used in many medical procedures, including laparoscopic procedures for example. These procedures often involve resecting portions or sections of tissue, followed by closing using staples. An exemplary procedure is colorectal anastomosis. In hybrid surgeries where physicians use laparoscopic and endoscopic platforms to conduct a procedure, a rigid stapler is often used. Linear staplers, which may include long rigid members, may be incapable of being navigated through tortuous anatomy without causing trauma to the tissue.

### SUMMARY OF THE DISCLOSURE

According to an example, a fastening mechanism includes an actuator, a fastening device attached to a distal end of the actuator, and a device coupler. The fastening device is pivotally attached to the device coupler.

In another example, the actuator may move longitudinally to pivot the fastening device relative to the device coupler. The fastening mechanism may further include a sheath, wherein the sheath covers a portion of the actuator, and the actuator is longitudinally movable relative to the sheath. The actuator may include at least one inner drive mechanism that actuates the fastening device to open and close the fastening device and/or deploy fasteners. The fastening device may be a stapler that includes a head and an anvil, and the head includes a stapler cartridge from which staples are held and deployed. The stapler cartridge may include a plurality of longitudinal slots from which a plurality of staples are deployed. The head of the stapler may be comprised of a flexible material that allows the head to flex laterally. A proximal end of the actuator may be connected to a controller that operates the fastening mechanism. The controller may operate longitudinally moving the actuator in either direction, opening the fastening device, and/or simultaneously deploying fasteners and closing the fastening device. The actuator may be comprised of a flexible material. The device coupler may include a proximal support fixed to a distal support, wherein the proximal support is fixed to the sheath. The fastening device may be pivotally attached to the distal support of the device coupler. The proximal support may include at least one flexible arm. The distal support may define a lumen with an open distal end or a clear barrier at the end of the lumen.

According to an example, a fastening mechanism includes an actuator, a stapler attached to a distal end of the actuator, a sheath in which the actuator moves longitudinally, and a device coupler for attaching the stapler to an outer surface of a distal end of a medical device. The device coupler includes a proximal support and a distal support, the proximal support is fixedly coupled to the sheath, and the stapler is pivotally attached to the distal support of the device coupler.

In another example, the medical device may be an endoscope, and the proximal support and the distal support may be fixed to a distal end of the endoscope. The actuator may be longitudinally movable relative to the sheath to pivot the stapler relative to the device coupler. The actuator may include at least one inner drive mechanism that actuates the stapler to open and close the stapler and/or deploy staples.

According to an example, a method of fastening tissue includes inserting an endoscope and a stapling mechanism fixed to an external surface of the endoscope, into a natural orifice of a subject, delivering the endoscope and the stapling mechanism to target tissue, pivoting the stapling mechanism relative to the distal end of the endoscope, positioning tissue between a head and an anvil of the stapling mechanism, and deploying a staple from the stapler mechanism to fasten the target tissue.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 is a perspective view of an endoscope according to an embodiment.

FIG. 2 is a perspective view of a stapling mechanism according to an embodiment.

FIG. 3 is a perspective view of the stapling mechanism coupled to an endoscope, according to an embodiment.

FIG. 4 is another perspective view of the stapling mechanism and endoscope of FIG. 3.

### DETAILED DESCRIPTION

Reference will now be made in detail to aspects of the present disclosure, examples of which are illustrated in the accompanying drawings. Wherever possible, the same or similar reference numbers will be used through the drawings to refer to the same or like parts. The term “distal” refers to a portion farthest away from a user when introducing a device into a subject (e.g., patient). By contrast, the term “proximal” refers to a portion closest to the user when placing the device into the subject.

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of +10% in a stated value or characteristic.

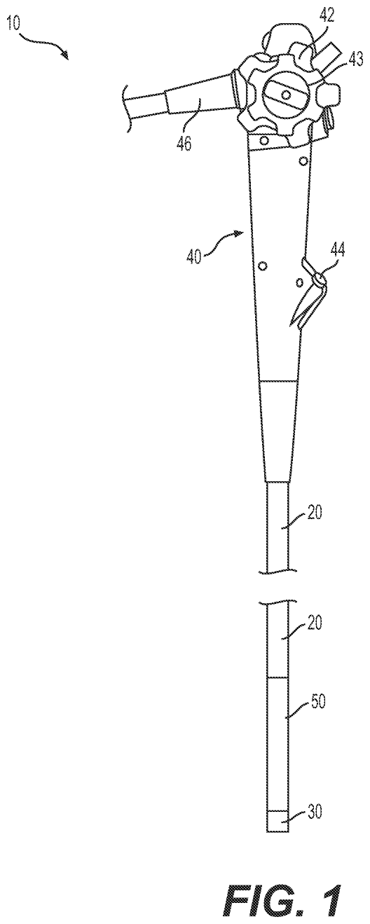

Endoscopic stapling may be especially useful in endoscopic, outpatient procedures. The present disclosure may solve one or more of the problems in the art. The scope of the disclosure, however, is defined by the attached claims and not the ability to solve a specific problem. The present disclosure is drawn to systems and devices, and related methods, for a stapling mechanism of a medical device, among other aspects. Referring to FIG. 1, an endoscope 10 according to an embodiment is shown. Endoscope 10 includes a flexible shaft 20, a tip 30 at a distal end of endoscope 10, and an articulation joint 50 disposed between and connecting flexible shaft 20 and tip 30. A handle 40 or some other device for actuating or controlling endoscope 10, and any tool or devices associated with endoscope 10, is connected at a proximal end of flexible shaft 20.

A plurality of actuating elements (not shown), such as cables or wires suitable for medical procedures (e.g., medical grade plastic or metal), may extend distally from a proximal end of endoscope 10 (such as handle 40) to articulation joint 50 and/or tip 30. For example, articulation/actuation wires may be indirectly coupled to first and second actuating devices 42, 43, which control articulation of articulation joint 50 in multiple directions, such as up, down, left, and right. Devices 42, 43, may be, for example, rotatable knobs that rotate about their axes to push/pull actuating elements.

Alternatively, or additionally, a user may operate actuating elements independently of handle 40. Distal ends of actuating elements extend through flexible shaft 20 and terminate at articulation joint 50 and/or tip 30. For example, one or more actuating elements may be connected to articulation joint 50 and one or more other actuating elements may be attached to tip 30. Actuation of actuating elements may control actuating joint 50, tip 30, and/or elements attached to tip 30, such as an end effector (not shown). In addition, one or more electrical cables (not shown) may extend from the proximal end of endoscope 10 (such as handle 40) to tip 30 and may provide electrical controls to imaging, lighting, and/or other electrical devices in or on tip 30, and may carry imaging signals from tip 30 proximally to be processed and/or displayed on a display. Handle 40 also may include a port 44 for introducing and/or removing tools, fluids, or other materials to and from the patient. A strain relief 46 attaches to an umbilicus for introducing fluid, suction, and/or wiring for electronic components. Endoscope 10 is an exemplary endoscope, in which a stapling mechanism may be attachable. The disclosure is not limited to a stapling mechanism for use in connection with endoscope 10. Any other medical scope (e.g. colonoscope, ureteroscope, duodenoscope, etc.), catheter, sheath, or the like, may be used in combination with the stapling mechanisms of this disclosure.

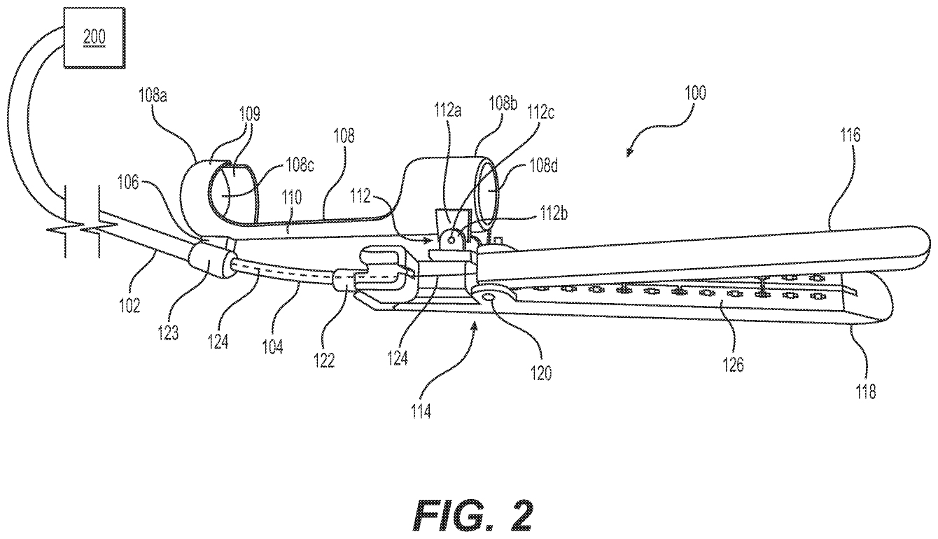

Referring to FIG. 2, an embodiment of a stapling mechanism 100 for a medical device, including, but not limited to, an endoscope, e.g., endoscope 10, is shown. Stapling mechanism 100 includes an actuator 104, a sheath 102, a device coupler 108, and a stapler 114.

Actuator 104 is a flexible tube, made of plastic or other suitable biocompatible, flexible material. Actuator 104 is sufficiently long to extend along a complete length of an endoscope, such as the flexible shaft 20 of endoscope 10, and sufficiently flexible to traverse tortuous anatomy of a patient. The distal end of actuator 104 is attached to the proximal end of stapler 114 via a stapler connector 122. The proximal end of actuator 104, at least a portion of which is sheathed by sheath 102, may be connected to any suitable controller or handle known in the art, represented by the box 200, at the proximal end of stapling mechanism 100.

Sheath 102 also is a flexible tube, made of plastic or other suitable biocompatible, flexible material. Sheath 102, in embodiments, is sufficiently long to extend along a complete length of an endoscope, such as the flexible shaft 20 of endoscope 10, and sufficiently flexible to traverse tortuous anatomy of a patient. The distal end of sheath 102 is fixedly attached to a sheath connector 106 at coupler 123. The sheath 102 may be coaxial with the actuator 104. Like actuator 104, the proximal end of sheath 102 also may be connected to any suitable controller or handle known in the art, e.g., box 200, at the proximal end of stapling mechanism 100, in such a manner to permit longitudinal sliding of actuator 104 within sheath 102. The controller may actuate the stapling mechanism to perform operations including, but not limited to, longitudinally moving actuator 104 in either direction (distally or proximally) by extending or pulling on actuator 104 relative to sheath 102. The controller also may control a stapling operation of stapler 114, to staple intended target tissue.

With continued reference to FIG. 2, device coupler 108 has a proximal support 108a, a distal support 108b, and a coupler arm 110 connecting proximal support 108a and distal support 108b. Proximal support 108a includes sheath connector 106 and two arms 109 that can wrap around a medical device including, for example, an endoscope shaft or articulation joint. In some embodiments, the arms 109 may be flexible, e.g., to elastically deflect, or flex apart from each other. Arms 109 define an opening 108c that accepts the medical device. Each arm 109 may deflect, or flex radially outward to accept the medical device, and be biased radially inward to rigidly couple to the medical device, e.g., to engage circumferentially around the endoscope 10. In other embodiments, proximal support 108a may be in different configurations that may also hold and support a medical device, including, but not limited to, a single flexible arm or a single ring. Sheath connector 106 fixedly attaches proximal support 108a to sheath 102. This may ensure a more stable linkage between device coupler 108 and sheath 102.

As shown in FIG. 2, distal support 108b is an annular, cap-like, ring defining a lumen 108d therein. Support 108b may hold and support a distal end of a medical device including, but not limited to, an endoscope tip. Distal support 108b may have an open distal end. In other embodiments, distal support 108b may have a distal end having a transparent, clear cover or barrier, serving as a cap over the distal face of the medical device tip. An open distal end or a clear cap on its distal end allows endoscope 10 to continue its various functions, such as providing a source of light and imaging via its camera, without being impeded. In other embodiments, distal support 108b may be substituted with other various supports, embodying different configurations that may also hold and support a medical device.

Proximal support 108a is connected to distal support 108b via coupler arm 110. Coupler arm 110 is grooved, or curved, to accommodate the shape of a medical device resting upon coupler arm 110. Distal support 108b is attached to a proximal end of stapler 114 via a pivot connection 112. Pivot connection 112 includes a distal support linking arm 112a, stapler linking arms 112b, and a pivot pin 112c that pins distal support linking arm 112a and stapler linking arms 112b together. Distal support linking arm 112a is fixedly attached to and extends from distal support 108b, while stapler linking arms 112b are fixedly attached to and extend from a proximal location of stapler 114. Both distal support linking arm 112a and stapler linking arms 112b have a pin hole. In pivot connection 112, distal support linking arm 112a and stapler linking arms 112b overlap with one another, so that distal support linking arm 112a lies between the arms of stapler linking arms 112b and their respective pin holes align, thereby allowing pivot pin 112c to be inserted and pin them together. Pivot connection 112 allows for the rotation of stapler 114, about pivot pin 112c and relative to device coupler 108, by the longitudinal movement of actuator 104 relative to sheath 102. For example, actuator 104 may be extended distally by, for example, an operation of a controller, e.g., box 200, to which it is attached. In another example, actuator 104 may be pulled proximally relative to sheath 102, resulting in stapler 114 rotating or pivoting in an opposite direction about the longitudinal axis of pivot pin 112c.

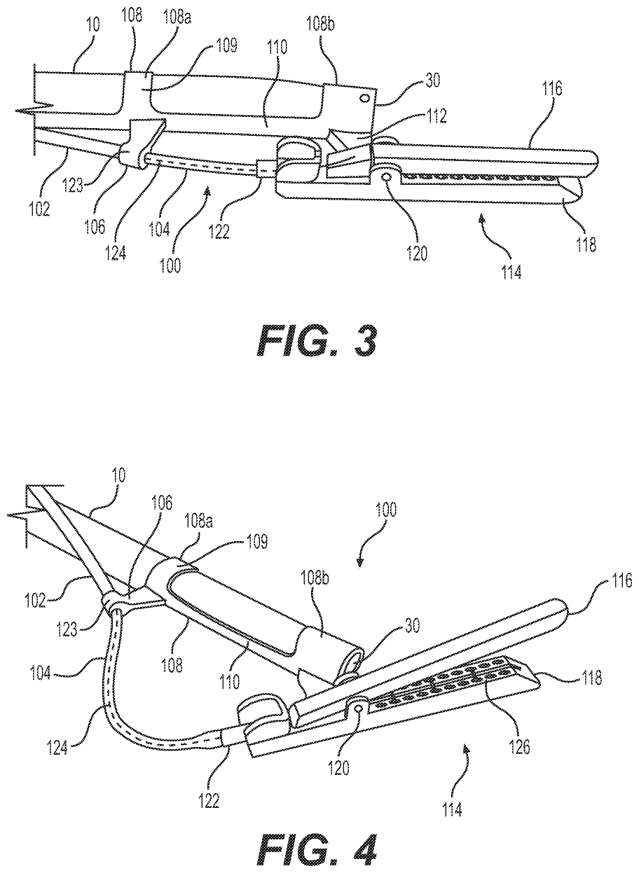

Referring to FIG. 3, proximal support 108a is fixed, via two arms 109, relative to a distal location of endoscope 10, such as, at articulation joint 50 (referring to FIG. 1). As shown, arms 109 receive endoscope 10, thereby coupling endoscope 10 to stapling mechanism 100. Fixing stapling mechanism 100 relative to a distal location of endoscope 10 via proximal support 108a provides a base from which actuator 104 extends towards stapler 114 to rotate stapler 114 in a clockwise or counter-clockwise direction.

FIG. 4 is another perspective view of the embodiment shown in FIG. 3. As shown, actuator 104 is extended distally, thereby pivoting stapler 114 about pivot connection 112 in a counter-clockwise direction. As evident from FIG. 4, the degree by which stapler 114 may be rotated clockwise or counter-clockwise may be adjusted based on the degree by which actuator 104 is moved longitudinally in either direction. For example, the further actuator 104 is extended distally, the further stapler 114 will pivot about pivot connection 112 in a counter-clockwise direction, until the rotation of stapler 114 is impeded by the presence of device coupler 108. Likewise, the further actuator 104 is pulled proximally, the further stapler 114 will pivot about pivot connection 112 and rotate in a clockwise direction, until the rotation of stapler 114 is impeded by the presence of device coupler 108.

FIG. 4 also illustrates stapler 114 in an open position. In the open position, the distal ends of an anvil 116 and a head 118 of stapler 114 are spaced apart, thereby forming a gap in which materials including, but not limited to, tissue may be held within and stapled together. In a closed position, inner surfaces of anvil 116 and head 118 are in a closest position relative to each other, so that very little gap, or no gap, exists between the inner surfaces of anvil 116 and head 118, as illustrated in FIG. 3. Stapler 114 can be opened or closed by anvil 116 pivoting about a stapler pivot 120, which holds anvil 116 and head 118 together at a proximal location of stapler 114.

Actuation of a drive mechanism opens and closes stapler 114. With reference to FIG. 4, a wire 124 is within a lumen of actuator 104, and the distal end of wire 124 connects to a proximal end of anvil 116. The proximal end of wire 124 may connect to a controller (not shown) at the proximal end of stapling mechanism 100. When wire 124 is pulled proximally, anvil 116 may rotate about stapler pivot 120, to open stapler 114. Pushing wire 124 distally may rotate anvil 116 about stapler pivot, to close stapler 114.

FIG. 4 further illustrates that head 118 includes a stapler cartridge 126, from which one or more staples may be held and deployed. The mechanism of deploying staples may be actuated by another drive mechanism (not shown), e.g. a second inner drive mechanism, within the lumen of actuator 104. The form of this second inner drive mechanism is not particularly limited, and it may be in the form of another wire (not shown) within actuator 104. The distal end of this second inner wire may connect to a mechanism with head 118 that will deploy one or more staples. The proximal end of the second inner wire may connect to a controller (not shown), which may trigger the second inner wire to actuate the deployment of a staple from head 118, by operation thereof. Inner wire 124 and the second inner wire (for deployment of staples) may work in conjunction, by operation of a controller, to simultaneously deploy a staple and close the stapler, thereby securely stapling an intended target, e.g., tissue. In some embodiments, wire 124 that opens and closes stapler 114 and the second drive mechanism for deploying one or more staples, may be operated from the same controller. In other embodiments, the different drive mechanisms may be operated by different controllers.

In an embodiment, head 118 may contain a plurality of staples in stapler cartridge 126, which may be deployed from stapler cartridge 126 when under the influence of a driving force exerted by an actuation sled (not shown), as disclosed in U.S. Patent Application No. 62/812,538, “Systems, Device, and Related Methods for Fastening Tissue”, filed Mar. 1, 2019, the contents of which are incorporated herein by reference in their entirety. Head 118 may have longitudinal slots (not shown) which may permit staples to pass through stapler cartridge 126 and pierce an intended target, e.g., tissue. In some embodiments, an actuation mechanism may be arranged to move proximally in a longitudinal direction from a distal end of stapler cartridge 126 toward a proximal end of cartridge 128 when actuated, contacting staples within stapler cartridge 126 and activating the staples through longitudinal slots, or a surface of the stapler cartridge, in order to couple staples to target tissue. Alternatively, in some embodiments, the actuation mechanism may be arranged to move distally in a longitudinal direction from a proximal end of stapler cartridge 126 toward a distal end of stapler cartridge 126 when actuated. In either manner, actuation mechanism may be actuated via the second drive mechanism, as discussed above. For example, actuation of a second inner wire in actuator 104, via a controller, may deploy staples into target tissue. In some examples, a single staple may extend through each slot. Each staple may be partially within the slot prior to deployment to assist with alignment of the staple with the slot. In some embodiments, two (or more) actuation mechanisms (not shown) may be required to actuate two (or more) different longitudinal rows of staples in stapler cartridge 126.

In some embodiments, head 118, including stapler cartridge 126, may be comprised of any appropriate biocompatible material, including flexible materials that may permit head 118 to flex laterally during insertion into, or positioning within, the body of the subject. Such flexibility may further permit stapler 114 to be carefully navigated through tortuous anatomy without causing trauma to surrounding tissue.

Anvil 116 may include a groove (not shown) positioned longitudinally that may align with the longitudinal slots of head 118, when stapler 114 is in a closed position (see FIG. 3). Once head 118 is actuated to deploy a staple and the legs of a deployed staple pierce through an intended target, the groove may act as a receiving base that effectively anvils the legs of a staple, as stapler 114 is being closed, so that the deployed staple is securely fastened to target tissue.

Referring to FIGS. 2-4, an example of a method of stapling an intended target, e.g., tissue, using the embodiment of a stapling mechanism illustrated in FIGS. 2-4 is further discussed below. A user may couple or attach stapling mechanism 100 to a medical device, e.g., endoscope 10, and deliver mechanism 100 and endoscope 10 into the body of a subject, e.g., via a natural orifice (such as a mouth or anus) and through a tortuous natural body lumen of the subject, such as an esophagus, stomach, colon, etc. A user may direct mechanism 100 and endoscope 10 to an intended target site by various means, including imaging. Once a target site is reached, a user may open stapler 114, if it is closed, by operation of a controller (not shown), which triggers wire 124 to pivot anvil 116 about stapler pivot 120. A user may then rotate stapler 114 in a clockwise or counter-clockwise direction by operation of the same or a different controller (not shown) that is attached to actuator 104, which may be moved longitudinally in either direction via the controller. It is noted that a user may also choose to rotate stapler 114 first, and then open stapler 114 after rotation thereof. The order of the stapler rotation and opening steps may be reversed and is not limited to a particular order. After positioning stapler 114 by rotating it, a user may manually maneuver the stapler or employ a mechanical means (not shown), including, but not limited to a grasper, suction, etc., that assists in placing the intended target, e.g., tissue, within the stapler, so that the intended target is positioned between head 118 and anvil 116 of stapler 114. A user may then staple the intended target by operating the same or a different controller, which triggers another drive mechanism, such as a wire, to actuate the deployment of a staple from stapler cartridge 126. Actuation of the controller may simultaneously deploy a staple and close the stapler, thereby stapling an intended target. The various mechanisms of stapling mechanism 100, including, but not limited to, rotation of stapler 114, opening/closing of stapler 114, and the deployment of staples from stapler cartridge 126, may be actuated by operation of the same controller, or different controllers.

It will be apparent to those skilled in the art that various modifications and variations can be made to the disclosed stapling mechanism without departing from the scope of the disclosure. For examples, the configuration of a coupler, actuator, and a stapler may be altered to suit any medical device, and are not limited to the examples described herein. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A medical assembly comprising:
an end effector;
a sheath; and
a device coupler, wherein the device coupler includes a proximal support, a distal support, and a coupler arm extending between the proximal support and the distal support, wherein the coupler arm includes a surface configured to accommodate a shape of a medical device resting upon the coupler arm, wherein the end effector is attached to the distal support, and wherein the proximal support is attached to the sheath.

2. The medical assembly according to claim 1, further comprising: an actuator, wherein device distal end of the actuator is attached to a portion of the end effector.

3. The medical assembly according to claim 2, wherein the actuator is longitudinally movable to pivot the end effector relative to the device coupler.

4. The medical assembly according to claim 2, wherein the actuator is longitudinally movable relative to the sheath.

5. The medical assembly according to claim 2, wherein the actuator includes at least one inner drive mechanism that actuates the end effector to (1) close the end effector or (2) deploy fasteners.

6. The medical assembly according to claim 1, wherein the device coupler removably attaches the end effector to an outer surface of a distal end of the medical device.

7. The medical assembly of claim 1, wherein the coupler arm joins the proximal support to the distal support.

8. The medical assembly of claim 1, wherein the end effector is pivotably attached to the distal support.

9. The medical assembly of claim 1, wherein the proximal support comprises at least one flexible arm.

10. The medical assembly of claim 2, wherein the sheath covers a portion of an actuator that is configured to actuate an aspect of the end effector.

11. A medical assembly comprising:
a sheath;
an end effector attached to a distal end of the sheath;
a device coupler for attaching the end effector to an outer surface of a distal end of a medical device, the device coupler including:
a proximal support coupled to the sheath;
a distal support coupled to the end effector and defining a lumen for receiving the distal end of the medical device; and
a coupler arm extending between the proximal support and the distal support.

12. The medical assembly according to claim 11, wherein the medical device is an endoscope.

13. The medical assembly according to claim 11, wherein the distal support is pivotably coupled to the end effector via a pivot pin.

14. The medical assembly of claim 13, wherein a first arm extends from the distal support, wherein a second arm extends from the end effector, and wherein the pivot pin extends through the first arm and the second arm.

15. The medical assembly of claim 11, wherein the proximal support includes a pair of proximal support arms that define an opening for receiving the distal end of the medical device.

16. The medical assembly of claim 11, wherein the distal support has an annular shape.

17. A medical assembly comprising:
a sheath,
an end effector attached to a distal end of the sheath; and
a device coupler including a proximal support coupled to the sheath, a distal support coupled to the end effector, and a coupler arm fixedly coupling the proximal support to the distal support, and wherein the coupler arm includes a curved surface.

18. The medical assembly according to claim 17, wherein the distal support is pivotably coupled to the end effector via a pivot pin.

19. The medical assembly of claim 18, wherein a first arm extends from the distal support, wherein a second arm extends from the end effector, and wherein the pivot pin extends through the first arm and the second arm.

20. The medical assembly of claim 18, wherein the proximal support includes a pair of proximal support arms that define an opening for receiving the distal end of the medical assembly, and wherein the distal support includes a ring.
